(19) United States
(12) Patent Application Publication
(10) Pub. No.: US 2025/0325839 A1
(43) Pub. Date: Oct. 23, 2025

(54) ULTRASOUND HISTOTRIPSY WITH FULLY SAMPLED TRANSDUCER ARRAY

(71) Applicant: ACOUSTIIC INC., Bellevue, WA (US)

(72) Inventors: Holly Lay, Seattle, WA (US); Paul Reynolds, Renton, WA (US)

(73) Assignee: ACOUSTIIC INC., Bellevue, WA (US)

(21) Appl. No.: 18/640,389

(22) Filed: Apr. 19, 2024

(51) Int. Cl. A61N 7/00; B06B 1/06

(52) U.S. Cl. CPC A61N 7/00; B06B 1/0629; A61N 2007/0039; A61N 2007/006; A61N 2007/0078; A61N 2007/0086; B06B 2201/76

(57) ABSTRACT

Systems and techniques are provided for generating histotripsy therapy pulses using full cycle transmit. An ultrasonic system may include a transducer array and amplifier electronics. The transducer array may include transducer elements. The transducer elements may be positioned on the transducer array such that the separation between neighboring transducer elements of the transducer elements in both elevation and azimuth is no more than one wavelength of the ultrasonic center frequency of the transducer array. The amplifier electronics may drive the transducer array.

### BACKGROUND

Histotripsy is the use of short, high intensity ultrasound waves to induce cavitation in a target media, such as tissue in a patient, resulting in mechanical damage to targeted tissue. Histotripsy may also use slightly longer pulses that may cause boiling in the targeted tissue and mechanical tissue destruction around the targeted tissue. Clinical and pre-clinical histotripsy systems may use specialty ultrasound transducers with either a single or a small number of active transducer elements that do not allow full electronic steering of the acoustic beam generated by the ultrasound transducers without a level of degradation of the acoustic beam that may have an impact on the clinical uses of the acoustic beam. These histotripsy systems may require mechanical translation to allow full access to the areas of a patient that are being treated, increasing the odds of de-registration, targeting error, and patient injury and possibly increasing the required treatment time.

### BRIEF DESCRIPTION OF THE DRAWINGS

The accompanying drawings, which are included to provide a further understanding of the disclosed subject matter, are incorporated in and constitute a part of this specification. The drawings also illustrate implementations of the disclosed subject matter and together with the detailed description serve to explain the principles of implementations of the disclosed subject matter. No attempt is made to show structural details in more detail than may be necessary for a fundamental understanding of the disclosed subject matter and various ways in which it may be practiced.

FIG. 1 shows an example system for ultrasound histotripsy with fully sampled transducer array according to an implementation of the disclosed subject matter.

FIG. 2 shows an example system for ultrasound histotripsy with fully sampled transducer array according to an implementation of the disclosed subject matter.

FIG. 3 shows an example system for ultrasound histotripsy with fully sampled transducer array according to an implementation of the disclosed subject matter.

FIG. 4 shows an example system for ultrasound histotripsy with fully sampled transducer array according to an implementation of the disclosed subject matter.

FIG. 5 shows an example system for ultrasound histotripsy with fully sampled transducer array according to an implementation of the disclosed subject matter.

FIG. 6 shows a computer according to an implementation of the disclosed subject matter.

FIG. 7 shows a network configuration according to an implementation of the disclosed subject matter.

### DETAILED DESCRIPTION

A fully sampled transducer array of an ultrasonic system may allow full electronic steering of the acoustic beam without mechanical translation of the transducer array, improved isolation of sensitive areas from insonification, and wave aberration correction. The fully sampled transducer array may be used to perform histotripsy on greater volumes of target organs or on organs such as the pancreas that may not otherwise be reachable using other ultrasound systems, may be usable in metal-hostile environments such as in magnetic resonance (MR) suites. A fully sampled transducer array may, for example, be an array of M transducers by N transducers that is fully sampled in both directions.

A fully sampled transducer array for ultrasound histotripsy may be sub-divided into transducer elements. The transducer elements may be individual ultrasonic transducers of any suitable type, including, for example, piezoelectric transducers. The transducer elements of a transducer array that is part of an ultrasonic system that uses translation steering for linear translation of the target zone may be separated by no more than one wavelength of the ultrasonic center frequency of the transducer array. The transducer elements of a transducer array that is part of an ultrasonic system that uses translation and rotation steering for angular and linear translation of the target zone may be separated by no more than half of a wavelength of the ultrasonic center frequency of the transducer array. In some implementations, the separation between transducer elements along the azimuth of the transducer array may be different from the separation between transducer elements along the elevation of the transducer array. For example, the transducer elements of the transducer array may be separated by ⅓ of a wavelength of the ultrasonic center frequency in one of azimuth and elevation and ⅔ of a wavelength of the ultrasonic center frequency in the other of azimuth and elevation. Using any of these separation distances between transducer elements may prevent the appearance of grating lobes, improve directivity for high angle steering, and may allow for electronic beam steering of the acoustic beam without, or with minimal, mechanical translation while allowing the acoustic beam to maintain high enough power levels to be used for histotripsy.

An acoustic beam generated by a fully sampled transducer array may allow for generation of a bubble cloud from cavitation behind or at the back of the target media, for example, targeted tissue, but in front of a sensitive area that should not be subjected to cavitation. This may allow for histotripsy to be used on a particular target, for example, tissue, without damaging non-target areas behind the target.

A fully sampled transducer array and the amplifier electronics used to drive the transducer elements along with cable and wiring connecting the transducer array and amplifier electronics may weigh in total less than 10 kg or may have a ratio of weight to active acoustic emission surface area of the transducer array of 1 g per mm².

A fully sampled transducer array may be used in conjunction with a disposable gel pad coupling or water bath or may be used with direct skin contact with a gel for coupling to the transducer array to a surface, for example, a patient's skin, in front of target area, for example a patient's tissue.

The ultrasound array may include transducer elements that form imagers, receiving reflected ultrasound waves. The transducer elements that form imagers may be arranged in 1:N format, for example in any number of locations on the transducer array, where N is the number of transducer elements in a row or column of the transducer array. Signals generated by the imagers may be used to generate images based on reflected ultrasound waves.

During operation of the ultrasonic system, transducer elements of the transducer array may all be fired to form the acoustic beam, or sub-aperture array beamforming may be used with any suitable subset of the transducer elements being fired to form acoustic beams.

Any suitable number of transducer elements, or subgroups of transducer elements, may have their own acoustic micro-lens. An acoustic micro-lens may be a fixed, removable, or interchangeable acoustic lens that may be used to focus ultrasonic waves generated by a transducer elements or subgroup of transducer elements of a transducer array.

The ultrasonic system with a fully sampled transducer array may be used for both therapy, for example, histotripsy, and for imaging. The ultrasonic system may be operated in either therapy or imaging modes, and may, for example, switch between the therapy and imaging modes as necessary during treatment. In therapy mode, the ultrasonic system may use the transducer array to generate acoustic beams with a power level and focus sufficient for histotripsy. In imaging mode, the ultrasonic system may use the transducer array to generate acoustic beams at a power level and focus for being reflected off of structures, such as tissue, in the area being imaged, to receive the reflections of the acoustic beams to generate an image. Imaging mode may also receive reflections from the therapy mode output to image.

In some implementations, robotic manipulation of the transducer array may be used to compensate for the slight degradation to the electronic beam steering when, for example, the separation of the transducer elements slightly deviates from being no more than one wavelength of the ultrasonic center frequency of the transducer array when using translation steering or no more than half of a wavelength of the ultrasonic center frequency of the transducer array when using translation and rotation steering. Movement of the transducer array may also be used when the targets are widely separated outside the full steering range of the transducer array or movement is desired to avoid a sensitive organ, nerve, blood vessel or other area, an air filled cavity such as colon, or existing implants such as metallic stomach staple.

The ultrasonic system may be used in conjunction with another imaging device in order to provide guidance and/or monitoring of the application of the acoustic beam. For example, the ultrasonic system may be used with another ultrasound device or with magnetic resonance (MR) device that may be used for imaging.

The acoustic beam generated using the transducer elements of the transducer array may act as the insonification of the target media, for example, tissue, and the imagers of the transducer array may receive signal reflection.

Other types of imagers may be used to detect the effects of the acoustic beam used for histotripsy as it moves through an object, such as a patient's body, to a target media. For example, the imager may be a magnetic resonance imager using elastography or may use optical measurement of the visible/surface return signals.

A measurement of the location of a low power focal point may be made using ultrasound from the fully sampled transducer array or other imaging such as MR, for example, elastography or thermography. This may allow detection of sub-therapeutic level changes before applying acoustic beams of a sufficient power level for histotripsy.

Robotic or manual positioning of the fully sampled transducer array may be used to move the fully sampled transducer array outside its electronic scanning volume or to provide a different angle of view.

The fully sampled transducer array may be constructed using any suitable and appropriate transmit electronics that may be capable of generating the electrical drive needed to generate an acoustic beam that can be used to perform histotripsy. The transmit electronics may be integrated application specific integrated circuits (ASICs), field programmable gate arrays (FPGAs), discrete analog circuitry, or digital drivers and control software, or any combination thereof. The transmit electronics may be located adjacent to or in contact with the ultrasound array or may be located any suitable distance from the ultrasound array and connected to the ultrasound array via wires or cables. The transmit electronics may allow for beam correction due to tissue heterogeneity, or aberration correction, amplitude correction to minimize energy delivery and correct generation of cavitation in the target media while reducing amplitudes below critical levels in sensitive areas near the target media, and/or the issuance of warnings that treatment of desired region, for example targeted tissue, while safely avoiding nearby regions, for example, non-targeted tissue, is not possible under existing circumstances.

The fully sampled transducer array may be integrated into a full therapy system that may include control hardware, software, monitoring devices such as an MRI, CT scanner, ultrasound system, biometric monitors, optical monitors, or any other suitable monitoring devices, operator interface, power systems and cooling systems.

The number of transducer elements fired may be reduced while still maintaining no grating lobes due to restricting angles. A sub-system of the ultrasound system may have the ability to adjust the drive amplitude on individual or sub-groups of transducer elements including the ability to turn individual or sub-groups of transducer elements off completely.

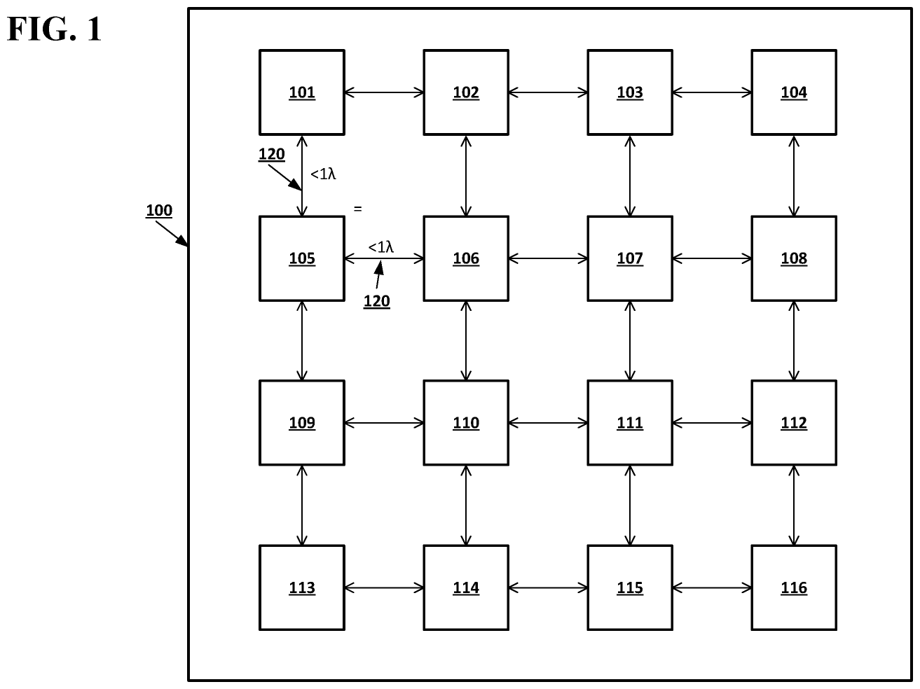

FIG. 1 shows an example system for ultrasound histotripsy with fully sampled transducer array according to an implementation of the disclosed subject matter. A transducer array 100 may include transducer elements 101, 102, 103, 104, 105, 106, 107, 108, 109, 110, 111, 112, 113, 114, 115, and 116, which may be any suitable type of transducers for generating ultrasonic acoustic waves. For example, the transducer elements 101-116 may be ultrasonic transducers of any suitable type, including piezoelectric transducers. Neighboring transducer elements of the transducer elements 101-116 of the transducer array 100 that is part of an ultrasonic system that uses translation steering for linear translation of the target zone may be separated by no more than one wavelength of the ultrasonic center frequency of the transducer array 100. The separation distance 120 may be the same in both the elevation and azimuth directions for all of the transducer elements 101-116 in the transducer array 100. The total size of the transducer array in elevation and azimuth, or equivalent in non-rectangular arrays, may be sufficient for the near/far field boundary of the acoustic beam at therapy frequency to be within the target region. The total size of a circular transducer array may be, for example, approximately (diameter\hat{ }2)/(4*wavelength), and the total size of a rectangular array may be up to approximately (1.4*(sidelength\hat{ }2)/(4*wavelength)).

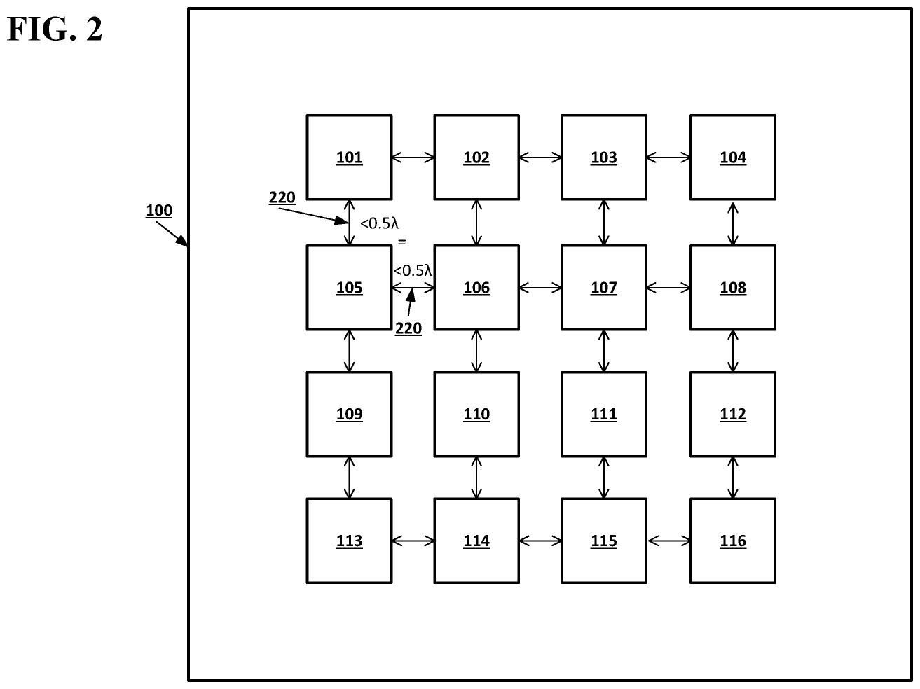

FIG. 2 shows an example system for ultrasound histotripsy with fully sampled transducer array according to an implementation of the disclosed subject matter. Neighboring transducer elements of the transducer elements 101-116 of the transducer array 100 that is part of an ultrasonic system that uses translation and rotation steering for angular and linear translation of the target zone may be separated by no more than half of a wavelength of the ultrasonic center frequency of the transducer array 100. The separation distance 220 may be the same in both the elevation and azimuth directions for all of the transducer elements 101-116 in the transducer array 100.

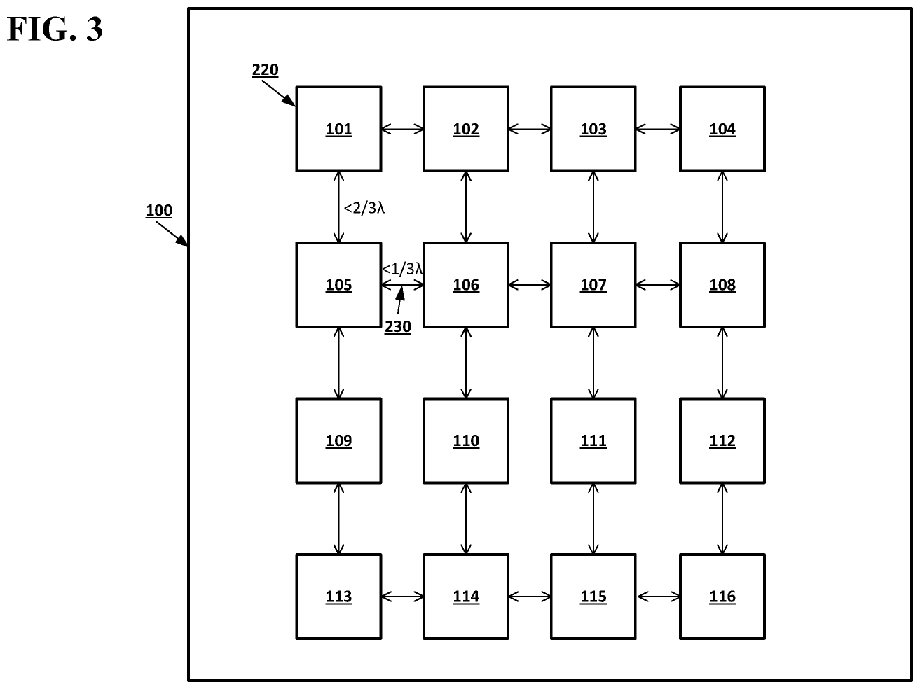

FIG. 3 shows an example system for ultrasound histotripsy with fully sampled transducer array according to an implementation of the disclosed subject matter. In some implementations, the separation between neighboring transducer elements of the transducer elements 101-116 along the azimuth of the transducer array 100 may be different from the separation between transducer elements 101-116 along the elevation of the transducer array 100. For example, the transducer elements 101-116 of the transducer array 100 may be separated by a separation distance 230 of ⅓ of a wavelength of the ultrasonic center frequency of the transducer array 100 in the azimuth direction and a separation distance 220 of ⅔ of a wavelength of the ultrasonic center frequency of the transducer array 100 in the elevation direction. Using these separation distances between the transducer elements 101-116 may prevent the appearance of grating lobes, and may allow for beam steering without, or with minimal, mechanical translation.

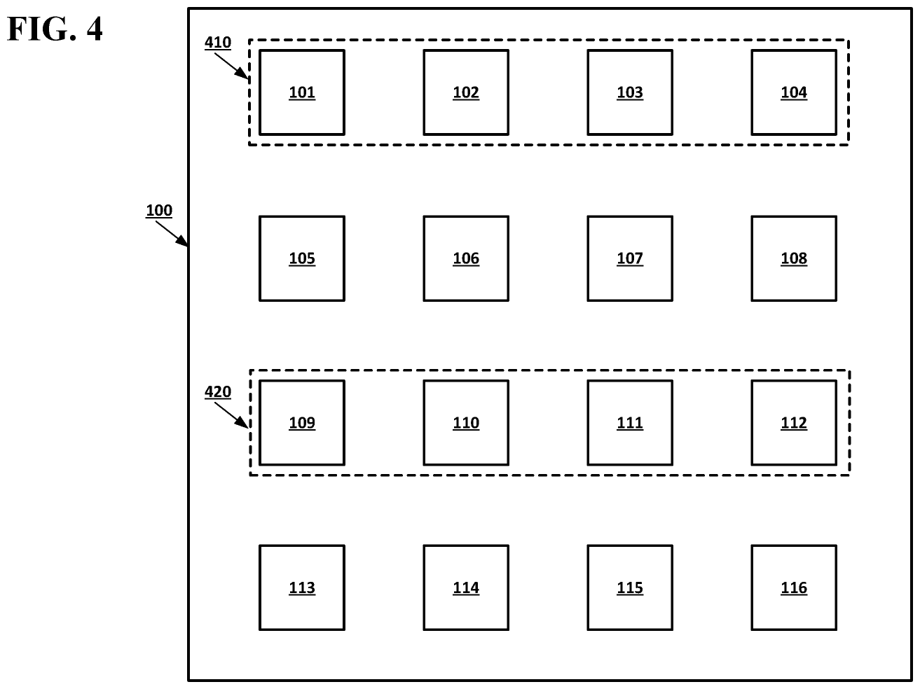

FIG. 4 shows an example system for ultrasound histotripsy with fully sampled transducer array according to an implementation of the disclosed subject matter. The transducer array 100 may include imagers 410 and 420. The imagers 410 and 420 may be formed from transducer elements arranged in 1:N format. The transducer elements that form imagers may be arranged in 1:N format in any number of locations on the transducer array 100. For example, the imager 410 may be a 1:4 imager formed from the transducer elements 101, 102, 103, and 104 and the imager 420 may be a 1:4 imager formed from the transducer elements 109, 110, 111, and 112. The imagers 410 and 420 may generate images based on reflected ultrasound waves that may have been generated by the transducer array 100. This may be reconfigurable between pulses to be active along either elevation or azimuth direction. In some implementations, imagers may be formed from transducer elements arranged in an M:N format.

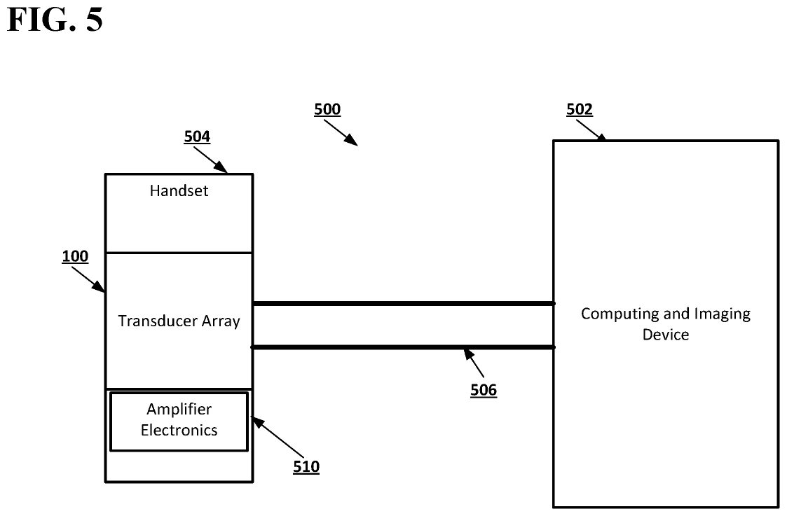

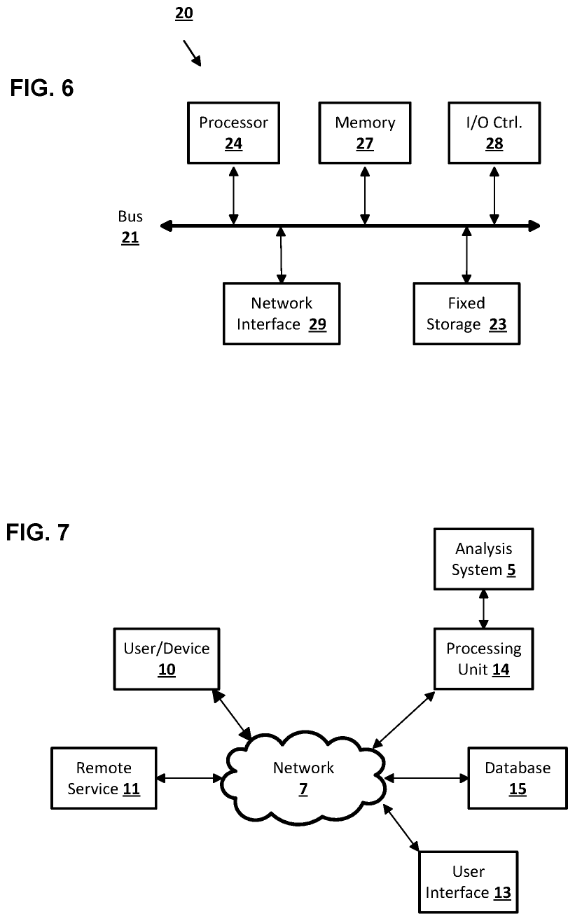

FIG. 5 shows an example system for ultrasound histotripsy with fully sampled transducer array. An ultrasound system 500 may include a handset 504 and a computing and imaging device 502 connected by a cable 506. The handset 504 may include a transducer array 100 that may include ultrasonic transducer elements arranged in an array. The cable 506 may allow for data to be transmitted in both directions between the handset 504 and the computing and imaging device 502. The cable 506 may also carry power to the handset 504. The computing and imaging device 502 may include any suitable computing hardware, running any suitable software, and any other suitable electronics to operate the ultrasound system 500, including supplying power and control signals to the transducer elements 101-116 of the transducer array 100, for example, through the cable 506, receiving signals from the transducer elements of the transducer array 100, performing any suitable computation to generate images from the signals received from the transducer elements 101-116 of the transducer array 100, and displaying generated images, for example, on a display directly connection to the computing and imaging device 502, or otherwise sending the generated images to a device, for example, a tablet or phone, that can display the generated images. The computing and imaging device 502 may have any suitable interface to allow a user to control the ultrasound system 500. The computing and imaging device 502 may be, or may include, a computer 20 as shown in in FIG. 6. The computing and imaging device 502 may also include any suitable electric and electronic components for delivering power to the handset 504 from any suitable power source, such as a battery or mains power. The handset 504 may also include amplifier electronics 510. The amplifier electronics 510 may be any suitable electronics for driving the transducer elements 101-116 of the transducer array 100 in the generation and steering of acoustic beams. The handset 504 of the ultrasound system 500, including the transducer array 100 and the amplifier electronics 510 (and connecting wiring or cabling) may weigh no more than 10 kg total, or less than 1 gram per square millimeter of acoustic emission surface area of the transducer array 100.

Implementations of the presently disclosed subject matter may be implemented in and used with a variety of component and network architectures. FIG. 6 is an example computer 10 suitable for implementations of the presently disclosed subject matter. The computer 10 includes a bus 11 which interconnects major components of the computer 10, such as a central processor 24, a memory 27 (typically RAM, but which may also include ROM, flash RAM, or the like), an input/output controller 28, a user display 22, such as a display screen via a display adapter, a user input interface 26, which may include one or more controllers and associated user input devices such as a keyboard, mouse, and the like, and may be closely coupled to the I/O controller 28, fixed storage 23, such as a hard drive, flash storage, Fibre Channel network, SAN device, SCSI device, and the like, and a removable media component 25 operative to control and receive an optical disk, flash drive, and the like.

The bus 11 allows data communication between the central processor 24 and the memory 27, which may include read-only memory (ROM) or flash memory (neither shown), and random access memory (RAM) (not shown), as previously noted. The RAM is generally the main memory into which the operating system and application programs are loaded. The ROM or flash memory can contain, among other code, the Basic Input-Output system (BIOS) which controls basic hardware operation such as the interaction with peripheral components. Applications resident with the computer 10 are generally stored on and accessed via a computer readable medium, such as a hard disk drive (e.g., fixed storage 23), an optical drive, floppy disk, or other storage medium 25.

The fixed storage 23 may be integral with the computer 10 or may be separate and accessed through other interfaces. A network interface 29 may provide a direct connection to a remote server via a telephone link, to the Internet via an internet service provider (ISP), or a direct connection to a remote server via a direct network link to the Internet via a POP (point of presence) or other technique. The network interface 29 may provide such connection using wireless techniques, including digital cellular telephone connection, Cellular Digital Packet Data (CDPD) connection, digital satellite data connection, or the like. For example, the network interface 29 may allow the computer to communicate with other computers via one or more local, wide-area, or other networks, as shown in FIG. 7.

Many other devices or components (not shown) may be connected in a similar manner (e.g., document scanners, digital cameras, and so on). Conversely, all of the components shown in FIG. 6 need not be present to practice the present disclosure. The components can be interconnected in different ways from that shown. The operation of a computer such as that shown in FIG. 6 is readily known in the art and is not discussed in detail in this application. Code to implement the present disclosure can be stored in computer-readable storage media such as one or more of the memory 27, fixed storage 23, removable media 25, or on a remote storage location.

FIG. 7 shows an example network arrangement according to an implementation of the disclosed subject matter. One or more clients 10, 11, such as local computers, smart phones, tablet computing devices, and the like may connect to other devices via one or more networks 7. The network may be a local network, wide-area network, the Internet, or any other suitable communication network or networks, and may be implemented on any suitable platform including wired and/or wireless networks. The clients may communicate with one or more servers 13 and/or databases 15. The devices may be directly accessible by the clients 10, 11, or one or more other devices may provide intermediary access such as where a server 13 provides access to resources stored in a database 15. The clients 10, 11 also may access remote platforms 17 or services provided by remote platforms 17 such as cloud computing arrangements and services. The remote platform 17 may include one or more servers 13 and/or databases 15.

More generally, various implementations of the presently disclosed subject matter may include or be implemented in the form of computer-implemented processes and apparatuses for practicing those processes. The disclosed subject matter also may be implemented in the form of a computer program product having computer program code containing instructions implemented in non-transitory and/or tangible media, such as floppy diskettes, CD-ROMs, hard drives, USB (universal serial bus) drives, or any other machine readable storage medium, wherein, when the computer program code is loaded into and executed by a computer, the computer becomes an apparatus for practicing implementations of the disclosed subject matter. Implementations also may be implemented in the form of computer program code, for example, whether stored in a storage medium, loaded into and/or executed by a computer, or transmitted over some transmission medium, such as over electrical wiring or cabling, through fiber optics, or via electromagnetic radiation, wherein when the computer program code is loaded into and executed by a computer, the computer becomes an apparatus for practicing implementations of the disclosed subject matter. When implemented on a general-purpose microprocessor, the computer program code segments configure the microprocessor to create specific logic circuits. In some configurations, a set of computer-readable instructions stored on a computer-readable storage medium may be implemented by a general-purpose processor, which may transform the general-purpose processor or a device containing the general-purpose processor into a special-purpose device configured to implement or carry out the instructions.

Implementations may use hardware that includes a processor, such as a general-purpose microprocessor and/or an Application Specific Integrated Circuit (ASIC) that embodies all or part of the techniques according to embodiments of the disclosed subject matter in hardware and/or firmware. The processor may be coupled to memory, such as RAM, ROM, flash memory, a hard disk or any other device capable of storing electronic information. The memory may store instructions adapted to be executed by the processor to perform the techniques according to embodiments of the disclosed subject matter.

The foregoing description, for purpose of explanation, has been described with reference to specific implementations. However, the illustrative discussions above are not intended to be exhaustive or to limit implementations of the disclosed subject matter to the precise forms disclosed. Many modifications and variations are possible in view of the above teachings. The implementations were chosen and described in order to explain the principles of implementations of the disclosed subject matter and their practical applications, to thereby enable others skilled in the art to utilize those implementations as well as various implementations with various modifications as may be suited to the particular use contemplated.

1. A device comprising:
a transducer array comprising transducer elements, wherein the transducer elements are disposed on the transducer array such that the separation between neighboring transducer elements of the transducer elements in both elevation and azimuth is no more than one wavelength of the ultrasonic center frequency of the transducer array.

2. The device of claim 1, wherein the transducer array uses translation steering.

3. The device of claim 1, wherein the separation between neighboring transducer elements of the transducer elements in both elevation and azimuth is no more than half of the wavelength of the ultrasonic center frequency of the transducer array.

4. The device of claim 3, wherein the transducer array uses translation and rotation steering.

5. The device of claim 1, wherein the separation between neighboring transducer elements of the transducer elements is different in elevation than in azimuth and is no more than two thirds of a wavelength of the ultrasonic center frequency of the transducer array.

6. The device of claim 1, wherein the transducer array uses the transducer elements to generate a steered acoustic beam for histotripsy.

7. The device of claim 1, wherein an imager of the transducer array comprises at least one group of the transducer elements arranged in a M:N form, wherein N is a number of transducer elements across either the width or the height of the transducer array and wherein M is at least 1.

8. The device of claim 1, further comprising at least one micro-lens.

9. An ultrasonic system comprising:
a transducer array comprising transducer elements, wherein the transducer elements are disposed on the transducer array such that the separation between neighboring transducer elements of the transducer elements in both elevation and azimuth is no more than one wavelength of the ultrasonic center frequency of the transducer array; and
amplifier electronics for driving the transducer array.

10. The system of claim 9, further comprising an imaging device, wherein the imaging device is a magnetic resonance imager or an ultrasound imager.

11. The system of claim 9, wherein the transducer array and amplifier electronics and cabling and wiring connecting the transducer array and amplifier electronics weigh less than 10 kg together or weigh less than 1 gram per square millimeter of acoustic emission surface area of the transducer array.

12. The system of claim 9, wherein gel pad coupling, water bath, or direct surface contact with gel is used to couple the transducer array to a surface.

13. The system of claim 9, further comprising a sub-system that adjusts the drive amplitude on individual ones of the transducer elements or sub-groups of the transducer elements and turns off completely individual ones of the transducer elements or sub-groups of the transducer elements.

14. The system of claim 9, wherein the transducer array is used to generate an acoustic beam for histotripsy.

15. The system of claim 14, wherein the transducer array is used as an imager during histotripsy by alternating the mode of operation of the ultrasonic system between therapy and imaging modes.

16. The system of claim 9, wherein the separation between neighboring transducer elements of the transducer elements in both elevation and azimuth is no more than half of the wavelength of the ultrasonic center frequency of the transducer array.

17. The system of claim 16, wherein the transducer array uses translation and rotation steering.

18. The system of claim 9, wherein the separation between neighboring transducer elements of the transducer elements is different in elevation than in azimuth and is no more than two thirds of a wavelength of the ultrasonic center frequency of the transducer array.

19. The system of claim 9, wherein the transducer array of the ultrasonic system generates an acoustic beam that causes generation of a bubble cloud behind or in back of a target area.

20. The system of claim 9, wherein the transducer array is positioned through robotic manipulation.
